(19) United States
(12) Patent Application Publication
(10) Pub. No.: US 2009/0069884 A1
(43) Pub. Date: Mar. 12, 2009

(54) STENT HAVING A BASE BODY OF A BIOCORRODABLE ALLOY

(75) Inventor: Heinz Mueller, Erlangen (DE)

Correspondence Address: BRYAN CAVE POWELL GOLDSTEIN, ONE ATLANTIC CENTER FOURTEENTH FLOOR, 1201 WEST PEACHTREE STREET NW, ATLANTA, GA, 30309-3488, US

(73) Assignee: BIOTRONIK VI PATENT AG, Baar (DE)

(21) Appl. No.: 12/204,957

(22) Filed: Sep. 5, 2008

(30) Foreign Application Priority Data
Sep. 6, 2007 (DE) 10 2007 042 451.7

(51) Int. Cl. A61F 2/06

(52) U.S. Cl. 623/1.46; 623/1.15; 623/1.44

(57) ABSTRACT

A stent comprising a base body consisting at least in part of either an arsenic-containing or selenium-containing biocorrodable alloy of at least one element selected from the group consisting of magnesium, iron, tungsten, zinc and molybdenum. A method for producing a stent with a base body having a core and a diffusion layer covering the core and an arsenic-containing and/or selenium-containing biocorrodable alloy comprising at least one element selected from the group consisting of magnesium, iron, tungsten, zinc and molybdenum.

### PRIORITY CLAIM

This patent application claims priority to German Patent Application No. 10 2007 042 451.7, filed Sep. 6, 2007, the disclosure of which is incorporated herein by reference in its entirety.

### FIELD

The present disclosure relates to a stent having a base body comprised entirely or in part of a biocorrodable alloy of the elements magnesium, iron, tungsten, zinc or molybdenum and a method for manufacturing such a stent.

### BACKGROUND

Implantation of stents has become established as one of the most effective therapeutic methods for treatment of vascular diseases. The purpose of stents is to assume a supporting function in a patient's hollow organs. Stents of a traditional design, therefore, have a tubular base body with a filigree supporting structure of metallic struts which are present initially in a compressed form for being introduced into the body and then are widened at the site of application. One of the main areas for use of such stents is for permanent or temporary widening of vascular occlusions and keeping the occlusions open, in particular, constrictions (stenoses) of the myocardial vessels. In addition, aneurysm stents are also known, for example, serving to support damaged vascular walls.

The base body of the stent consists of an implant material. An implant material is a nonviable material which is used for an application in medicine and interacts with biological systems. The basic prerequisite for use of a material as an implant material which comes in contact with the body's environment when used as intended, is its compatibility with the body (biocompatibility). For purposes of the present disclosure, the term biocompatibility refers to the ability of a material to induce an appropriate tissue reaction in a specific application. This includes adaptation of the chemical, physical, biological and morphological surface properties of an implant to the receiving tissue with the goal of a clinically desired interaction. The biocompatibility of an implant material also depends on the chronological sequence of the reaction of the biosystem into which the implant is implanted. Irritation and inflammation may thus occur in the relatively short term and may lead to tissue changes. Biological systems react in different ways depending on the properties of the implant material. The implant materials may be subdivided into bioactive, bioinert and degradable/resorbable materials depending on the reaction of the biosystem. For the purposes of the present disclosure, only metallic implant materials for stents are of interest, more specifically biocorrodable alloys of the elements magnesium, iron, tungsten, zinc or molybdenum.

A biological reaction to metallic elements depends on concentration, duration of action and method of administration. The presence of an implant material alone often leads to inflammation reactions, the trigger being mechanical stimuli, chemicals or metabolites. The inflammation process is usually accompanied by migration of neutrophilic granulocytes and monocytes through the vascular walls, migration of lymphocyte effector cells, forming specific antibodies to the inflammation stimulus, activation of the complement system with the release of complement factors that act as mediators and, ultimately, the activation of blood coagulation. An immunological reaction is usually closely associated with the inflammation reaction and can lead to sensitization and allergization. One important problem with stent implantation in a blood vessel is in-stent restenosis due to excessive neointimal growth which is induced by a strong proliferation of arterial smooth muscle cells and a chronic inflammation reaction.

It is known that a higher measure of biocompatibility and thus an improvement in restenosis rate can be achieved if metallic implant materials are provided with coatings of especially tissue-compatible materials. These materials are usually of an organic or synthetic polymer type and may be of natural origin in some cases. Additional strategies to prevent restenosis are concentrated on inhibiting proliferation by medication, e.g., treatment with cytostatics. However, such coatings also influence the degradation of the stent from the biocorrodable alloy so that extensive test methods and optimization procedures are required. Adaptation of the stent, e.g., with a change in the design, the coating system or the alloy composition requires a renewed run-through of the test methods and optimization procedures. In addition, production and handling of such coated stents are complex and thus associated with high costs.

Despite the advances that have been achieved, a further integration of the stent into its biological environment and, therefore, a reduction in restenosis rate would be desirable. In addition, there is a demand for simplification of the production and handling of the stent.

### SUMMARY

The present disclosure describes several exemplary embodiments of the present invention.

One aspect of the present disclosure provides a stent, comprising a base body consisting at least in part of either an arsenic-containing or selenium-containing biocorrodable alloy of at least one element selected from the group consisting of magnesium (Mg), iron (Fe), tungsten (W), zinc (Zn) and molybdenum (Mo).

Another aspect of the present disclosure provides a method for producing a stent with a base body having a core of a biocorrodable alloy comprising at least one element selected from the group consisting of magnesium, iron, tungsten, zinc and molybdenum, the alloy not containing arsenic or selenium; and a diffusion layer of the biocorrodable alloy containing at least one material selected from the group consisting of arsenic and selenium covering the core, the method comprising (i) providing a base body of the stent of a biocorrodable alloy comprising at least one element selected from the group consisting of magnesium, iron, tungsten, zinc and molybdenum, the alloy not containing arsenic or selenium; (ii) contacting the surface of the base body with either arsenic or selenium or in either bound or elemental form; and (iii) simultaneously with step (ii) or following step (ii), thermally treating the stent at least in the area of the contact surface so as to form a diffusion layer that contains arsenic or selenium.

A further aspect of the present disclosure provides an arsenic-containing biocorrodable alloy, comprising the elements magnesium, iron, tungsten, zinc or molybdenum.

An additional aspect of the present disclosure provides a selenium-containing biocorrodable alloy, comprising the elements magnesium, iron, tungsten, zinc or molybdenum.

### DETAILED DESCRIPTION

A first aspect of the present disclosure provides a stent having a base body consisting entirely or in part of a biocorrodable alloy of the elements magnesium, iron, tungsten, zinc or molybdenum containing arsenic and/or selenium. Such a stent solves or improves at least one or more of the problems described hereinabove.

The present disclosure is based on the finding that, in a healthy body, there is an equilibrium between cellular proliferation and cellular death (apoptosis). If restenosis occurs after stent implantation, the equilibrium between these two processes is disturbed and proliferation gains the upper hand over natural cell death. Previous strategies to prevent restenosis have relied on inhibition of proliferation. However, histological preparations of stenosed vessels have not provided evidence of any elevated concentration of proliferation markers in comparison with the surrounding tissue. This supports the assumption that apoptosis takes place less effectively here than in healthy tissue. This is where the present invention begins. The imbalance is to be compensated by increasing the rate of apoptosis. One advantage in comparison with inhibiting proliferation includes, among other things, preventing an accumulation of neointimal cells without delaying required tissue coverage of the stent which is often observed when using proliferation-inhibiting substances, such as sirolimus or paclitaxel.

It has now surprisingly been found that the use of selenium and/or arsenic as a component of the biocorrodable alloy of the base body of the stent leads to increased apoptosis. The mechanism of action on which the positive influence of arsenic and/or selenium on apoptosis is based is still largely unelucidated. Presumably, the caspase-3 enzyme, which is involved in the apoptotic process, is activated.

The base body of the stent is gradually degraded after implantation. Since the pharmacologically active elements are very homogeneously distributed in the alloy, the resulting local release profile is accordingly very uniform in different sections of the stent.

If the alloy contains only arsenic and no selenium, then the arsenic content of the alloy in the Mg—As system is preferably 0.01-40 wt %, in particular, 0.01-20 wt %, and in the Fe—As, Mo—As, W—As and Zn—As systems, the arsenic content is preferably 0.01-20 wt %, in particular, 0.01-10 wt %. The arsenic content in the aforementioned alloys is especially preferably greater than 0.5 wt %, in particular, greater than 1 wt %.

If the alloy contains only selenium and no arsenic, then the selenium content of the alloy in the Mg—Se system is preferably 0.01-60 wt %, in particular, 0.01-30 wt %, and in the Fe—Se, Mo—Se, W—Se and Zn—Se systems, the selenium content is 0.01-30 wt %, in particular, 0.01-15 wt %. The selenium content in the aforementioned alloys is especially preferably greater than 0.5 wt %, in particular, greater than 1 wt %.

It is also preferable if the arsenic and selenium contents in the alloy in the Mg—As—Se system are 0.01-30 wt % As and 0.01-40 wt % Se, preferably 0.01-15 wt % As and 0.01-30 wt % Se. The arsenic content and the selenium content in the aforementioned alloys are especially preferably greater than 0.05 wt %, in particular, greater than 1 wt %.

Furthermore, it is also preferable if the arsenic content and the selenium content in the alloy in the Fe—As—Se, Mo—As—Se, W—As—Se and Zn—As—Se systems are 0.01-10 wt % As and 0.01-15 wt % Se, preferably 0.01-5 wt % As and 0.01-10 wt % Se. The arsenic content and the selenium content in the aforementioned alloys are especially preferably greater than 0.5 wt %, in particular, greater than 1 wt %.

A weight ratio of As to Se in the aforementioned arsenic-containing and selenium-containing alloys is preferably in the range of 1:100 to 100:1, preferably 1:100 to 10:1. It has been found that a combination of selenium and arsenic lowers the toxicity so that adverse effects are reduced.

Both As and Se form intermetallic phases with Mg, Fe, Mo, W and Zn. These phases are usually very brittle and thus greatly limit the processing as well as also, in particular, all applications in which the implant must be shaped (e.g., stent). The upper limits are selected so that at most half of the metallic matrix consists of brittle phases so the required shapability is retained for the purposes of the present disclosure.

The base body of the stent optionally comprises a biocorrodable magnesium alloy, a biocorrodable iron alloy, a biocorrodable tungsten alloy, a biocorrodable zinc alloy or a biocorrodable molybdenum alloy. For purposes of the present disclosure, the terms magnesium alloy, iron alloy, zinc alloy, molybdenum alloy and tungsten alloy refer to a metallic structure whose main component is magnesium, iron, zinc, molybdenum or tungsten, respectively. The main component is the alloy component which is present in the alloy in the greatest amount by weight. The amount of the main component is preferably greater than 50 wt %, in particular, greater than 70 wt %.

For purposes of the present disclosure, the term biocorrodable refers to alloys in which degradation/rearrangement takes place in a physiological environment so that the part of the implant comprising the material is entirely or at least predominately no longer present. The alloy is thus to be selected in its composition so that it is biocorrodable. The test medium for testing the corrosion behavior of an alloy in question is synthetic plasma as described according to EN ISO 10993-15:2000 for biocorrosion tests (composition NaCl 6.8 g/L, CaCl₂ 0.2 g/L, KCl 0.4 g/L, MgSO₄ 0.1 g/L, NaHCO₃ 2.2 g/L, Na₂HPO₄ 0.126 g/L, NaH₂PO₄ 0.026 g/L). A sample of the alloy to be tested is stored with a defined amount of the test medium at 37° C. in a sealed sample container. The samples are removed at intervals, based on the corrosion behavior to be expected, of a few hours up to several months and are examined for traces of corrosion by methods known in the art. The artificial plasma according to EN ISO 10993-15:2000 corresponds to a blood-like medium and thus presents a possibility for reproducibly adjusting a physiological environment.

The base body preferably comprises a biocorrodable magnesium alloy. The biocorrodable magnesium alloy especially preferably contains yttrium and other rare earth metals in addition to arsenic and/or selenium because such an alloy is excellent based on its physicochemical properties and high biocompatibility, in particular, including its degradation products. For purposes of the present disclosure, the general term “rare earth metals” includes scandium (21), yttrium (39), lanthanum (57) and the next 14 elements following lanthanum (57), namely cerium (58), praseodymium (59), neodymium (60), promethium (61), samarium (62), europium (63), gadolinium (64), terbium (65), dysprosium (66), holmium (67), erbium (68), thulium (69), ytterbium (70) and lutetium (71).

Magnesium alloys with the composition 5.2-9.9 wt % rare earth metals, including 3.7-5.5 wt % yttrium in which arsenic and/or selenium are present in the preferred amounts by weight indicated hereinabove are especially preferred, with magnesium claiming the complementary amount of alloy up to a total of 100 wt %.

According to another exemplary embodiment which can be implemented, in particular, in combination with the aforementioned exemplary embodiments, the base body of the stent comprises (a) a core of a biocorrodable alloy of the elements magnesium, iron, tungsten, zinc or molybdenum not containing arsenic or selenium; and (b) a diffusion layer of the biocorrodable alloy containing arsenic and/or zinc covering the core.

Such a diffusion layer has a very high ability to adhere to the core so that after mechanical deformation of the base body of the stent during use as intended damage to the diffusion layer need not be expected. The diffusion layer is created by a suitable processing method by deposition of arsenic or selenium on the core and simultaneous reaction of a portion of the core that is near the surface and is made of the metallic biocorrodable implant material with at least portions of this deposit. In other words, the applied arsenic or selenium forms the diffusion layer together with a portion of the metallic implant material near the surface. In the wake of the production process, the diffusion layer is formed due to diffusion processes at the phase boundary between the metallic material and the deposit containing arsenic or selenium. The developing alloy system of the diffusion layer depends on many factors, in particular, the temperature and treatment time in the production process, the composition of the core and the composition of the material containing arsenic or selenium used to create the diffusion layer.

The concentration of arsenic or selenium in the diffusion layer preferably declines from the outside of the stent toward the core so that the desired pharmacological effect of the two elements is established at a point in time soon after implantation, but further forcing of apoptosis is avoided after the healing process has begun.

The diffusion layer preferably has a layer thickness in the range of 20 nm to 50 μm, in particular 20 nm to 10 μm.

The core preferably comprises a biocorrodable magnesium alloy. For purposes of the present disclosure, the term magnesium alloy is a metallic structure whose main component is magnesium. The main component is the alloy component which is present in the greatest amount by weight in the alloy. The main component is preferably present in an amount greater than 50 wt %, in particular, greater than 70 wt %. The biocorrodable magnesium alloy of the core preferably contains yttrium and other rare earth metals because such an alloy is characterized by its physicochemical properties and high biocompatibility, in particular, also its degradation products. A magnesium alloy with the composition 5.2-9.9 wt % rare earth metals, including 3.7-5.5 wt % yttrium and <1 wt % remainder is especially preferred, whereby magnesium accounts for the remaining amount of the alloy up to a total of 100 wt %. This magnesium alloy has already confirmed its special suitability in clinical trials, i.e., it has a high biocompatibility, favorable processing properties, good mechanical characteristics and adequate corrosion properties for use purposes. In addition, magnesium alloys containing up to 6 wt % zinc are preferred. Furthermore, a magnesium alloy having the following composition is especially preferred: 0.5-10 wt % yttrium, 0.5-6 wt % zinc, 0.05-1 wt % calcium, 0-0.5 wt % manganese, 0-1 wt % silver, 0-1 wt % cerium and 0-1 wt % zirconium or 0-0.4 wt % silicon, whereby the amounts are based on the percentage of the alloy by weight, and magnesium and impurities due to the production process account for the remaining amount of the alloy up to a total of 100 wt %.

Another aspect of the present disclosure provides a method for producing a stent having a base body which (a) comprises a core of a biocorrodable alloy of the elements magnesium, iron, tungsten, zinc or molybdenum not containing arsenic or selenium; and (b) a diffusion layer covering the core and consisting of the biocorrodable alloy containing arsenic and/or selenium. The method includes the steps: (i) providing a base body of the stent of a biocorrodable alloy of the elements magnesium, iron, tungsten, zinc or molybdenum and not containing arsenic or selenium; (ii) bringing the surface of the base body in contact with selenium or arsenic in bound or elemental form; and (iii) simultaneously with step (ii) or following step (ii), thermally treating the stent at least in the area of the contact surface, forming a diffusion layer containing arsenic or selenium.

According to the method of the present disclosure, a stent of a biocorrodable metallic implant material is finished by creating a diffusion layer of parts of an implant material and selenium and/or arsenic.

The contact in step (ii) may be achieved by means of a CVD or PVD process, a flame-spray process or an electrolysis process. However, contact is preferably achieved by immersion in a solution or dispersion containing arsenic or selenium.

Step (iii) is preferably performed in such a way that the concentration of arsenic or selenium in the resulting diffusion layer decreases from the outside of the stent toward the core. This diffusion layer then has a concentration gradient for arsenic and/or selenium, i.e., the atomic fraction/weight of the elements in the alloy forming the diffusion layer decreases toward the core. The layer thickness of the diffusion layer formed depends first on the amount of arsenic or selenium applied and secondly on the extent of the reaction of the applied material with the metallic implant material of the stent in the area of the implant surface near the surface.

A biocorrodable alloy containing selenium or arsenic and the elements magnesium, iron, tungsten, zinc or molybdenum can be produced with traditional metallurgical methods. For example, these elements may be alloyed by melt metallurgy or by producing suitable powder mixtures which are then extruded, sintered or shaped in some other way to form the semifinished product. With all the aforementioned production methods, i.e., even the diffusion method described already for producing a diffusion layer, arsenic and/or selenium is present in the form of atoms dissolved interstitially in the lattice of the base metal or the base alloy or in the form of fine intermetallic precipitates. In this way, the homogeneous distribution and/or the set concentration gradient is achieved in the volume of the stent.

Such biocorrodable alloys of the elements magnesium, iron, tungsten, zinc or molybdenum containing arsenic and/or selenium have not been described previously in the art. Therefore, another aspect of the present disclosure also lies in providing the aforementioned alloys.

The present invention is explained in greater detail below on the basis of exemplary embodiments.

### EXAMPLES

### Example 1

Coating of an implant, in particular, a stent whose base body consists of a biodegradable iron or magnesium alloy with selenium may be performed as described below.

The stent is introduced into a chamber with a vacuum of at least 133.322×10⁻⁶ Pa (10⁻⁶ mmHg) as freely as possible, suspended from an electrically conducting holder. In this chamber, there is also a selenium target on a carrier which can be heated. An electric voltage can be applied between the carrier and the stent.

To perform the process, first the vacuum chamber is flooded with an inert gas (e.g., argon) to approximately 133.322×10⁻³ Pa (approximately 10⁻³ mmHg). Then a voltage is applied between the stent and the carrier for the selenium target and the selenium target is heated to approximately 200±25° C.

Due to the applied voltage, the selenium vaporized from the target is deposited on the stent. With a process duration of approximately 1 hour, layer thicknesses between 5 μm and 20 μm can thus be achieved, depending on the applied voltage and the target temperature.

Then in an annealing oven under an argon atmosphere, the stent with the layer thereby applied is annealed for a period of 1 to 72 hours at 450-525° C. (Mg alloys) and/or 500-750° C. (iron alloys), depending on the specific alloy. It should be noted that selenium has a melting point of approximately 220° C. On the other hand, however, even a very small amount of the foreign elements, specifically iron and/or magnesium here, leads to a very marked increase in the respective solidus temperatures. Thus, if a small amount of iron or magnesium is dissolved due to interdiffusion or if diffusion of magnesium or iron atoms into the molten phase of the selenium coating is made possible by keeping the stent above its melting point for a very short period of time, then diffusion may take place between the solid coating phase and the base body. Through such a calcining treatment, diffusion layers with a thickness up to approximately 50 μm can be obtained. The layers are usually designed as gradient layers. With shorter calcining times, a portion of the coating remains on the surface as pure selenium, apart from the impurities due to the manufacturing process. This may be desired if high doses of selenium are to be released immediately after implantation. With very long calcining times, the selenium diffuses completely. Some of the applied selenium is then always lost to this system due to sublimation.

If a target of arsenic or an arsenic-selenium alloy is used in the coating process described above (sputtering process), then an arsenic or arsenic-selenium diffusion layer is obtained in the same way. Such arsenic/arsenic-selenium targets with an arsenic content between 0.01 and 25 wt % are already being used commercially in the production of semiconducting layers. The process parameters can essentially also be applied here. Because of the higher melting point of arsenic (approximately 390° C.), the process parameters in diffusion annealing must be adapted accordingly, i.e., the temperature must usually be increased.

For deposition of the primary arsenic layer, so-called currentless coating processes in electrolytic baths containing arsenic, in which Ti³⁺ ions are used as reducing agents, may also be used for deposition of the primary arsenic layer; the stent is immersed in these baths for a period of time between five minutes and one hour. Such baths are available industrially. Arsenic layer thicknesses in a range between a few μm up to 25 μm can be produced. The subsequent diffusion annealing may be performed as described hereinabove.

To produce alloys of iron or magnesium containing arsenic and/or selenium, a traditional metallurgical melt method may be employed. However, it has proven advisable to use As—Mg, As—Fe, Se—Mg and/or Se—Fe pre-alloys in melting to prevent excessive evaporation of the arsenic and/or selenium components. The casting temperatures should be at least 200° C. above the liquidus temperatures of the respective alloys. Because of the low solubility of the elements in Mg and Fe (an exception is As—Fe system), the alloys should solidify as rapidly as possible to achieve the highest possible homogeneity; e.g., in a water-cooled chill mold or by casting in a cooled crucible or a cooled metal mold.

What is claimed is:

1. A stent, comprising: a base body consisting at least in part of either an arsenic-containing or selenium-containing biocorrodable alloy of at least one element selected from the group consisting of magnesium (Mg), iron (Fe), tungsten (W), zinc (Zn) and molybdenum (Mo).

2. The stent of claim 1, wherein the amount of arsenic in the alloy is 0.01-40 wt % in the Mg—As alloy and is 0.01-20 wt % in the Fe—As, Mo—As, W—As and Zn—As alloys.

3. The stent of claim 1, wherein the amount of selenium in the alloy is 0.01-60 wt % in the Mg—Se alloy and is 0.01-30 wt % in the Fe—Se, Mo—Se, W—Se and Zn—Se alloys.

4. The stent of claim 1, wherein the amounts of arsenic and selenium in the alloy are 0.01-30 wt % As and 0.01-40 wt % Se in the Mg—As—Se alloy and are 0.01-10 wt % As and 0.01-15 wt % Se in the Fe—As—Se, Mo—As—Se, W—As—Se and Zn—As—Se alloys.

5. The stent of claim 4, wherein a weight ratio of arsenic to selenium in the alloy is in the range of 1:100 to 100:1.

6. The stent of claim 1, wherein the base body comprises:
(a) a core of a biocorrodable alloy of at least one element selected from the group consisting of magnesium, iron, tungsten, zinc and molybdenum, the alloy not containing arsenic or selenium; and
(b) a diffusion layer of the biocorrodable alloying containing at least one material selected from the group consisting of arsenic and selenium covering the core.

7. The stent of claim 6, wherein a concentration of either arsenic or selenium in the diffusion layer decreases from the outside of the implant toward the core.

8. The stent of claim 6, wherein the diffusion layer has a thickness in the range of 20 nm to 50 μm.

9. A method for producing a stent with a base body having a core of a biocorrodable alloy comprising at least one element selected from the group consisting of magnesium, iron, tungsten, zinc and molybdenum, the alloy not containing arsenic or selenium; and a diffusion layer of the biocorrodable alloy containing at least one material selected from the group consisting of arsenic and selenium covering the core, the method comprising:
(i) providing a base body of the stent of a biocorrodable alloy comprising at least one element selected from the group consisting of magnesium, iron, tungsten, zinc and molybdenum, the alloy not containing arsenic or selenium;
(ii) contacting the surface of the base body with either arsenic or selenium or in either bound or elemental form; and
(iii) simultaneously with step (ii) or following step (ii), thermally treating the stent at least in the area of the contact surface so as to form a diffusion layer that contains arsenic or selenium.

10. The method of claim 9, wherein step (iii) is performed such that a concentration of either arsenic or selenium in the resulting diffusion layer decreases from the outside of the stent to the core.

11. An arsenic-containing biocorrodable alloy, comprising at least one element selected from the group consisting of magnesium, iron, tungsten, zinc and molybdenum.

12. A selenium-containing biocorrodable alloy, comprising at least one element selected from the group consisting of magnesium, iron, tungsten, zinc and molybdenum.
